(19) United States
(12) Patent Application Publication
(10) Pub. No.: US 2012/0016027 A1
(43) Pub. Date: Jan. 19, 2012

(54) Composition and Method for Improved Lens Comfort

(75) Inventor: Mark Willcox, Balmain (AU)

(73) Assignee: BRIEN HOLDEN VISION INSTITUTE, Sydney (AU)

(21) Appl. No.: 13/184,053

(22) Filed: Jul. 15, 2011

(30) Foreign Application Priority Data
Jul. 15, 2010 (AU) 2010903162

(51) Int. Cl. A61K 31/202; A61P 27/02

(52) U.S. Cl. 514/560

(57) ABSTRACT

The present invention relates to methods of preventing ocular discomfort, improving ocular comfort or ameliorating decrease in ocular comfort during wearing of an ophthalmic device (such as a contact lens), that include the administration of compositions comprising fatty acids, such as omega-3 and/or -6 fatty acids. The present invention also relates to fatty acid compositions suitable for use in the methods of the present invention.

### FIELD OF THE INVENTION

The invention relates to improving the comfort of wearing contact lenses or other similar ophthalmic devices. In particular, the invention is directed towards wearers with no other abnormalities of the eye such as eye disease, dry eye or allergic reactions (e.g. allergic conjunctivitis). The invention is directed towards a method for improving the comfort of the wearer, or ameliorating (by avoiding or minimising) decrease in ocular comfort, during the wearing of contact lenses.

### BACKGROUND OF THE INVENTION

A significant obstacle to contact lens wear for many people is comfort. Putting aside people who have pre-existing eye conditions that reduce the comfort of contact lens wear, the wearing of contact lenses can cause discomfort in otherwise healthy eyes.

Contact lenses in use today fall into two general categories. Hard type lenses are formed from materials prepared by the polymerization of acrylic esters, such as poly(methyl methacrylate) (PMMA). Gel, hydrogel or soft type lenses are made by polymerizing such monomers as 2-hydroxyethyl methacrylate (HEMA) or, in the case of extended wear lenses, made by polymerizing silicon-containing monomers or macromonomers.

Soft contact lenses and extended wear lenses typically contain water, usually 38 to 60% or higher. Evaporation of water from within the contact lens causes the contact lens to replenish this water by absorption of water from the tear layer of the eye. If the tear layer is dehydrated the contact lens becomes uncomfortable and vision is compromised. Contact lens wearers make up 90 to 100% of people using artificial tears/natural tears/rewetting drops. These medicaments are used by contact lens wearers to alleviate the discomfort associated with wearing contact lenses. Studies show that while these medicaments do lubricate, the effect lasts only a few minutes.

Moreover, in people who have a disorder resulting in an excessively curved cornea or protrusion of the cornea, wearing a contact lens can be an important part of treatment. This is not feasible however if it causes significant discomfort. Also, children are more likely to object to contact lens wear if it is uncomfortable, although it may be preferable for at least some children to wear contact lenses rather than glasses or no eye-correction at all.

It has been found that omega-3 and/or omega-6 fatty acids are useful in treating eye conditions and disorders such as Dry Eye Syndrome (DES). DES (sometimes also referred to as “dry eye disorder”) is a disorder of the normal tear film that results from one of the following:
decreased tear production;
excessive tear evaporation;
an abnormality in the production of mucus or lipids normally found in the tear layer.

Aqueous (watery) tear deficiency is caused by either poor production of watery tears or excessive evaporation of the watery tear layer. Poor production of tears by the tear glands may be a result of age, hormonal changes, or various autoimmune diseases, such as primary Sjogren syndrome, rheumatoid arthritis, or lupus. Evaporative loss of the watery tear layer is usually a result of an insufficient overlying lipid layer (which may be caused by, for example, a deficiency in oil production from the meibomian glands). In addition, some medications, such as antihistamines, antidepressants, beta-blockers, and oral contraceptives, may decrease tear production. Therefore, DES is a pathological condition.

US 2006/009522 relates to topical ophthalmic compositions containing omega-3 and/or omega-6 fatty acids for treating, among other eye diseases, DES. U.S. 7029712 relates to compositions containing a blend of omega-3 and omega-6 fatty acids, as well as nutrient co-factors that support the conversion of linoleic acid to gamma-linolenic acid, and the use of these compositions for the treatment of the underlying inflammatory processes that cause DES. WO 2007/130960 relates to self-emulsifying, oil-in-water compositions comprising omega-3 fatty acids and surfactants, which are to be used as a contact lens care solution to treat DES. Rashid, S. et al “Topical omega-3 and omega-6 fatty acids for treatment of dry eye”, Arch Ophthalmol (2008) 126(2), pages 219-25 discusses a study of the efficacy of topical application of omega-3 and -6 fatty acids for the treatment of DES. FR 2882895 relates to a composition for use as a food supplement, which includes, inter alia, linoleic acid and gamma-linolenic acid. This document also discusses that mixtures of linoleic acid and gamma-linolenic acid have been shown to significantly improve symptoms associated with DES. Kokke, K. H. et al “Oral omega-6 essential fatty acid treatment in contact lens associated dry eye”, Cont Lens Anterior Eye (2008) 31(3), pages 141-6 relates to a study conducted to investigate the effects of oral treatment with primrose oil (as a source of omega-6 fatty acids) on contact-lens associated DES.

The discomfort caused by wear of contact lenses (“contact lens intolerance”) is a condition caused by the interaction between a contact lens and the eye, and can be distinguished from DES, which is a pathological condition. Contact lens intolerance can usually be alleviated by removing the contact lens. Eye discomfort and vision impairment caused by contact lens intolerance is a prevalent problem in eye care for contact lens wearers.

Therefore, there is a need for compositions and methods that can be used to prevent, treat and/or ameliorate ocular discomfort associated with wearing of ophthalmic devices (such as contact lenses) in people who have healthy eyes i.e. people who do not have pre-existing eye conditions or diseases such as dry eye disorder (i.e. DES), recent eye surgery or other irritation or trauma, conjunctivitis, glaucoma and/or other allergy-related eye discomfort. Ideally, the compositions and methods should be sufficiently simple in their application such that a person can safely and effectively self-administer the compositions and methods, without requiring input from a specialist (such as a doctor).

Reference to any prior art in the specification is not, and should not be taken as, an acknowledgment or any form of suggestion that this prior art forms part of the common general knowledge in any jurisdiction or that this prior art could reasonably be expected to be ascertained, understood and regarded as relevant by a person skilled in the art.

### SUMMARY OF THE INVENTION

The present invention relates to the new insight that, by administering compositions including omega-3 and/or omega-6 fatty acids to people (particularly those with no pre-existing eye conditions) who wear, or who desire to wear, ophthalmic devices (such as contact lenses), the discomfort associated with wearing of the ophthalmic devices can be ameliorated or prevented.

The present invention provides, in one embodiment, a method of improving ocular comfort or ameliorating decrease in ocular comfort during wearing of an ophthalmic device (such as a contact lens), the method comprising administering to a person desiring to wear the ophthalmic device, a composition containing a therapeutically effective amount of an omega-3 compound, or a precursor thereof. In one embodiment, the omega-3 compound includes one or more of alpha linolenic acid (ALA), eicosapentaenoic acid (EPA) and docosahexaenoic acid (DHA). In another embodiment, the person does not have, prior to this administration, any one or more of (1) dry eye disorder, (2) recent eye surgery or other irritation or trauma, (3) conjunctivitis, (4) glaucoma or (5) another allergy related eye discomfort. In some embodiments, the person is first assessed as being in need of improved ocular comfort or ameliorated decrease in ocular comfort during wearing of an ophthalmic device. Typically, the person will have suffered some decrease in ocular comfort wearing a contact lens before administration. This is usually assessed subjectively by patients self-assessing comfort levels in the morning and evening. In one embodiment, the person is not taking an omega-3 supplement to their normal diet prior to the administration described above.

In one particular embodiment, the ophthalmic device is a contact lens. The device in other embodiments is any device that is physically in contact with the eye but readily removable.

The administration is preferably oral for simplicity. Another preferable administration is topical directly to the eye. Formulations suitable for oral administration and administration to the eye are described further below.

In one embodiment, the omega-3 compound, or a precursor thereof, is administered at a dose of between about 100 and 3000 mg per day. In another embodiment, the dose is between about 100 and 750 mg per day. For example, the dose may be about 750 mg per day. In another embodiment, the composition is administered in the form of one or more unit doses. For example, three capsules having a combined dose of 750 mg of omega-3 compound, or a precursor thereof, may be administered once a day to achieve a desired daily dose of omega-3 compound.

Optionally omega-6, or a precursor thereof, is also administered. In an alternative embodiment, no omega-6, or precursor thereof, is administered.

In another embodiment, the present invention provides a composition for improving ocular comfort or ameliorating decrease in ocular comfort during wearing of an ophthalmic device (such as a contact lens), comprising an omega-3 compound, or a precursor thereof. In one embodiment, the omega-3 compound includes one or more of alpha linolenic acid (ALA), eicosapentaenoic acid (EPA) and docosahexaenoic acid (DHA). In another embodiment, the composition is used on a person who does not have, prior to this administration, any one or more of (1) dry eye disorder, (2) recent eye surgery or other irritation or trauma, (3) conjunctivitis, (4) glaucoma or (5) another allergy related eye discomfort. The composition may also be used on a person who is first assessed as being in need of improved ocular comfort or ameliorated decrease in ocular comfort during wearing of an ophthalmic device. In another embodiment, the composition is used on a person who is not taking an omega-3 supplement to their normal diet prior to the administration. In one embodiment, the composition is formulated for oral administration. In other embodiments, the composition is formulated as a contact lens care solution, or for topical administration directly to the eye. The composition may also contain an omega-6 compound, or a precursor thereof. In an alternative embodiment, the composition contains no omega-6 compound, or precursor thereof. In yet another embodiment, the composition consists essentially of an omega-3 compound, or a precursor thereof.

The present invention also relates to a composition consisting essentially of one active component for improving ocular comfort or ameliorating decrease in ocular comfort during wearing of an ophthalmic device (such as a contact lens), wherein the active is an omega-3 compound, or a precursor thereof. The composition is formulated for oral administration. In another embodiment, there is provided a composition including omega-3 formulated as a contact lens care solution. In this specification “consists essentially of” means, in respect of the composition, that the composition contains only one active.

The present invention also relates to a method of improving ocular comfort or ameliorating decrease in ocular comfort during wearing of a contact lens, comprising administration of a therapeutically effective amount of an omega-3 compound, or a precursor thereof. The present invention also relates to the use of a therapeutically effective amount of an omega-3 compound, or a precursor thereof, for improving comfort or ameliorating decrease in ocular comfort during wearing of a contact lens. The present invention also provides a pharmaceutical composition for use in improving ocular comfort or ameliorating decrease in ocular comfort during wearing of a contact lens/ophthalmic device, in any of the embodiments described in the specification. The present invention also relates to the use of a therapeutically effective amount of an omega-3 compound, or a precursor thereof, for the manufacture of a medicament for improving ocular comfort or ameliorating decrease in ocular comfort during wearing of a contact lens/ophthalmic device. In one embodiment, the omega-3 compound, or a precursor thereof, is essentially the only active ingredient of the composition.

The present invention also relates to an omega-3 compound, or a precursor thereof, when used in a method of improving comfort or ameliorating decrease in ocular comfort during wearing of a contact lens/ophthalmic device. The present invention also relates to a composition having an active ingredient for use in improving comfort or ameliorating decrease in ocular comfort during wearing of a contact lens/ophthalmic device, wherein the active ingredient is an omega-3 compound, or a precursor thereof. In one embodiment, the omega-3 compound, or precursor thereof, is essentially the only active ingredient of the composition.

Further to the embodiments provided above, the composition (or omega-3 compound or precursor thereof) may be administered or used for a period of time prior to wearing of the ophthalmic device. Therefore, the present invention also relates to a method of preventing ocular discomfort associated with wearing of an ophthalmic device, the method comprising administering to a person desiring to wear the ophthalmic device, a composition containing a therapeutically effective amount of an omega-3 compound, or a precursor thereof. The period of time is preferably about four to eight weeks. The present invention also relates to the use of a composition comprising a therapeutically-effective amount of an omega-3 compound, or a precursor thereof, or to the use of a therapeutically effective amount of an omega-3 compound, or a precursor thereof, for preventing ocular discomfort associated with wearing of an ophthalmic device, In one embodiment, the omega-3 compound, or precursor thereof, is essentially the only active ingredient of the composition.

The present invention also relates to the use of an ophthalmic device containing an omega-3 compound in improving comfort or ameliorating decrease in ocular comfort during wearing of a contact lens/ophthalmic device, such as described above.

The present invention also relates to a method of making an ophthalmic device for improving comfort or ameliorating decrease in ocular comfort during wearing of a contact lens/ophthalmic device comprising a therapeutically effective amount of an omega-3 compound, comprising the step of contacting an ophthalmic device with a solution comprising the omega-3 compound during manufacture such that the omega-3 compound is released during wear.

In one embodiment the ophthalmic device is a contact lens. Preferably, the contact lens is a soft contact lens. The lens may be prepared by soaking the lens in a solution containing an omega-3 compound. Typically, the lens is soaked in the solution for 15 mins to 8 hours, preferably for 1 hour. The lens may also be prepared by a) omega-3 incorporated liposomes that are attached to the lens surface and b) a care solution formulation of an omega-3 compound being incorporated into the packaging solution in the case of daily disposable lenses. The omega-3 compound is desirably present in the solution in an amount ranging from 0.01 to 10% weight by volume. In one embodiment, it is present in an amount ranging from 0.1 to 5% weight by volume. In one embodiment, the omega-3 compound is essentially the only active ingredient of the solution.

In another embodiment there is provided a kit for use in a method of the invention mentioned above, the kit including:
a container holding an omega-3 compound, or a precursor thereof, or pharmaceutical composition of the invention; and
a label or package insert with instructions for use.

Optionally, the kit includes a vessel for containing a contact lens soaking in the composition.

In a further embodiment there is provided a kit when used in a method of the invention mentioned above, the kit including:
a container holding an omega-3 compound, or a precursor thereof, or pharmaceutical composition of the invention; and
a label or package insert with instructions for use.

In certain embodiments the kit may contain one or more further ingredients for preventing discomfort, improving comfort or ameliorating decrease in ocular comfort during wearing of a contact lens/ophthalmic device.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 shows the average comfort rating of participants in the morning and evening during the lens wear period of the trial, assessed as discussed herein.

FIG. 2 shows the “comfort” ratings as assessed on the same basis.

FIG. 3 shows the “ocular dryness” ratings as assessed on the same basis.

### DETAILED DESCRIPTION OF THE EMBODIMENTS

The term “preventing ocular discomfort” refers to averting, delaying or reducing in frequency and intensity the signs and/or symptoms associated with ocular discomfort (such as dryness and irritation) in a person desiring to wear an ophthalmic device. The term “preventing” is used herein in a clinical sense to mean inhibit discomfort occurring, rather than in an absolute sense of making it impossible for the discomfort to ever occur in a given subject. Therefore, inhibition of progression to discomfort or reduced new discomfort amounts to “prevention” within the meaning of this specification even if there is pre-existing discomfort. The term “improving ocular comfort” refers to increasing the ocular comfort of a person wearing an ophthalmic device by decreasing symptoms such as dryness and irritation. The term “ameliorating decrease in ocular comfort” refers to avoiding or minimising an increase in ocular discomfort of a person wearing an ophthalmic device.

The term “omega-3” or “omega-3 compound” refers to fatty acids that have double bonds three carbon atoms from their omega carbon atom. For example, an omega-3 fatty acid includes, but is not limited to, alpha linolenic acid (ALA). Other omega-3 fatty acids include derivatives of ALA. A “derivative” of ALA is a fatty acid that is made by a chemical modification performed upon ALA by, for example, an enzyme or is done by organic synthesis. Examples of omega-3 fatty acids that are derivatives of ALA include, but are not limited to, eicosapentaenoic acid (EPA), docosahexaenoic acid (DHA), and the like. An “omega-3 compound” can comprise one or more omega-3 fatty acids. Also useful for the invention are other precursors to omega-3 fatty acids i.e. compounds that are metabolised in the body to produce omega-3 fatty acids. The amount of omega-3 compound in the compositions of the present invention is typically from 0.01 to 10% weight by total volume in the case of a topical composition and 30 to 80% weight by total weight in the case of an oral composition (i.e. 30 to 80% w/w).

The term “omega-6” or “omega-6 compound” refers to one or more fatty acids that have a double bonds 6 carbon atoms from their omega carbon atoms. For example, an omega-6 fatty acid includes, but is not limited to linoleic acid (LA). Other omega-6 fatty acids include derivatives of LA. A “derivative” of LA is a fatty acid that is made by a chemical modification performed upon LA. Examples of omega-6 fatty acids that are derivatives of LA include, but are not limited to, gammalinolenic acid (GLA), dihomogammalinolenic acid (DGLA), arachidonic acid (AA), docosatetraenoic acid, and the like. An “omega-6 compound” can comprise one or more omega-6 fatty acids. Also useful are precursors to omega-6 fatty acids i.e. compounds that are metabolised in the body to produce omega-6 fatty acids. The amount of omega-6 compound in the compositions of the present invention is typically from 0.01 to 10% weight by total volume in the case of a topical composition and 30 to 80% weight by total weight in the case of an oral composition (i.e. 30 to 80% w/w). The compositions may contain no omega-6 compound.

The terms “therapeutically effective amount” or “effective amount” refer to an amount of an omega-3 compound, or a precursor thereof, that results in an improvement or remediation of the symptoms of ocular discomfort, prevents ocular discomfort, improves ocular comfort or ameliorates decrease in ocular comfort.

The term “pharmaceutical composition” or “composition” refers to a composition comprising an omega-3 compound, or a precursor thereof, which is dispersed in a pharmaceutically acceptable carrier. The composition may further include one or more additional excipients, such as diluents, emulsifiers, buffers, stabilizing agents, binders, fillers, and the like. The composition may also include omega-6.

The term “ophthalmic device” refers to an object that resides in or on the eye. The device may provide optical correction, physical correction (e.g. excessively curved or protruding cornea), or may be cosmetic. Ophthalmic devices include but are not limited to soft contact lenses, intraocular lenses, overlay lenses, ocular inserts, punctual plugs, and optical inserts. The preferred ophthalmic devices of the invention are soft contact lenses made from silicone elastomers or hydrogels, which include but are not limited to silicone hydrogels, and fluorohydrogels. The ophthalmic devices may be “single-use” devices e.g. single-use contact lenses.

Topical administration includes placing the omega-3 composition or ophthalmic device containing an omega-3 compound, or a precursor thereof, onto the surface of the eye, or in the eye, of a subject. Typically such a device (e.g. a soft contact lens) is in contact with the anterior surface of the subject's eye for eight to 16 hours daily. Alternatively, the omega-3 compound, or precursor thereof, may be placed into or onto an ocular insert as a method of drug delivery. Typically such an ocular insert is inserted into the space between the lids and the sclera (formix) and gradually releases the drug. Alternatively, a biodegradable collagen lens soaked in an omega-3 compound, or precursor thereof, may be placed onto the surface of the eye, or inserted into the eye, of a subject. Typically the collagen lens slowly dissolves and improves patient symptoms. The composition may also be administered to the subject by giving the subject a composition of the invention to consume orally.

As used herein, except where the context requires otherwise, the term “comprise” and variations of the term, such as “comprising”, “comprises” and “comprised”, are not intended to exclude further additives, components, integers or steps.

The pharmaceutical composition of the present invention may be an ophthalmic composition, which is a topical composition suitable for administration directly to the eye. Examples of ophthalmic compositions according to the invention are suspensions, ointments, emulsions, sustained release formulations, gels or solutions suitable for application as an eye drop.

Preferably, the pharmaceutical compositions according to the present invention will be formulated for oral administration, including optionally as sustained release delivery. Alternatively, the composition of the present invention is in a form suitable for administration to the eye e.g. an eye drop, a spray or by release from a soft contact lens. In another embodiment, the composition is administered other than by release from a contact lens.

Aqueous solutions are generally preferred for topical administration, based on ease of formulation, as well as a subject's ability to easily administer such compositions by means of instilling one to two drops of the solutions in the affected eyes. However, the compositions may also be suspensions, viscous or semi-viscous gels, or other types of solid or semi-solid compositions, or those appropriate for sustained release.

The “solutions” that are used in methods of this invention may be water-based (i.e. aqueous) solutions. Typical solutions include saline solutions, other buffered solutions, and deionized water. The preferred aqueous solution is deionized water or saline solution containing salts including sodium chloride, sodium borate, sodium phosphate, sodium hydrogenphosphate, sodium dihydrogenphosphate, or the corresponding potassium salts of the same. These ingredients are generally combined to form buffered solutions that include an acid and its conjugate base, so that addition of acids and bases cause only a relatively small change in pH. The buffered solutions may additionally include 2-(N-morpholino)ethanesulfonic acid (MES), sodium hydroxide, 2,2-bis(hydroxymethyl)-2,2′,2″-nitrilotriethanol, n-tris(hydroxymethyl)methyl-2-aminoethanesulfonic acid, citric acid, sodium citrate, sodium carbonate, sodium bicarbonate, acetic acid, sodium acetate, ethylenediamine tetraacetic acid and the like and combinations thereof. Preferably, the solution is a borate buffered or phosphate buffered saline solution or deionized water. The particularly preferred solution contains about 8 g/L NaCl, 0.2 g/KCl, 1.15 g/L Na₂HPO₄ and 0.2 g/L KH₂PO₄ buffer.

Any of a variety of carriers may be used in the compositions of the present invention including water, mixtures of water and water-miscible solvents, such as C₁- to C₇-alkanols, vegetable oils or mineral oils comprising from 0.5 to 5% non-toxic water-soluble polymers, gelling products, such as gelatin, alginates, pectins, tragacanth, karaya gum, xanthan gum, carrageenan, agar and acacia, and their derivatives, starch derivatives, such as starch acetate and hydroxypropyl starch, cellulose and its derivatives and also other synthetic products, such as polyvinyl alcohol, polyvinylpyrrolidone, methyl ether, polyethylene oxide, preferably cross-linked polyacrylic acid, such as neutral Carbopol, or mixtures of those polymers, naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

For the adjustment of the pH, preferably to a physiological pH, buffers may especially be useful. The pH of the present solutions should be maintained within the range of 4 to 8. It will be understood by a person of ordinary skill in the art that any pH that is compatible with the ocular surface is suitable. Suitable buffers may be added, such as boric acid, sodium borate, potassium citrate, citric acid, sodium bicarbonate, TRIS, disodium edetate (EDTA) and various mixed phosphate buffers (including combinations of Na₂HPO₁, NaH₇PO₄ and KH₂PO₄) and mixtures thereof. Generally, buffers will be used in concentrations ranging from about 0.05 to 0.5 M.

Tonicity is adjusted if needed typically by tonicity enhancing agents. Such agents may, for example, be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids.

In certain embodiments, the compositions of the invention additionally comprise a preservative. A preservative may typically be selected from a quaternary ammonium compound such as benzalkonium chloride (N-benzyl-N—(C₈-C₁₈alkyl)-N,N-dimethylammonium chloride), benzoxonium chloride or the like. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®π, Purite™ or sorbic acid. Preferred preservatives are quaternary ammonium compounds, in particular benzalkonium chloride or its derivative such as Polyquad (see U.S. Pat. No. 4,407,791), mercury salts and parabens. Where appropriate, a sufficient amount of preservative is added to the ophthalmic composition to ensure protection against secondary contaminations during use caused by bacteria and fungi.

Other compounds may also be added to the compositions of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

The composition of the present invention may also be in a form suitable for oral administration. For example, the composition may be administered in the form of a capsule or as an oil (e.g. fish oil). Typical compositions suitable for oral administration may include, in addition to the omega-3 compound, one or more carriers, diluents, solubilizers, and the like, including glycerol and vitamin E. For example, a suitable composition for oral administration comprises, per capsule, EPA in an amount of 450 mg, DHA in an amount of 300 mg, flaxseed oil in an amount of 1000 mg, vitamin E in an amount of 183 IU, a mixed tocopherol concentrate in an amount of 20 mg, and gelatin, glycerin and purified water. Such a composition is commercially available as TheraTears™ Nutrition for Dry Eye with omega-3. Another example of a suitable composition for oral administration comprises, per capsule, fish oil in an amount of 1000 mg (comprising EPA in an amount of 180 mg and DHA in an amount of 120 mg), glycerin and purified water. An example of an oil suitable for oral administration comprises, per 5 mL of the oil composition, fish oil in an amount of 4.6 g, which comprises EPA in an amount of 1.9 g and DHA in an amount of 927 mg. Another example of a fish oil suitable for oral administration comprises, per 5 mL of the oil composition, fish oil in an amount of 4.7 g, which comprises EPA in an amount of 1.7 g and DHA in an amount of 1.1 g.

The composition of the present invention and another active ingredient (e.g. an omega-6 compound) may be administered at the same time (either in the same or different compositions) or at times close enough such that the administration results in an overlap of the desired effect. Alternatively, the composition of the present invention may precede or follow other treatments. An example of a suitable omega-6-containing composition for oral administration comprises, per capsule, Evening Primrose Oil in an amount of 500 mg and fish oil in an amount of 500 mg, which comprises EPA in an amount of 90 mg and DHA in an amount of 60 mg.

The composition may be administered in any way that is deemed suitable by a person of ordinary skill in the art. The pharmaceutical composition may be administered topically. The composition of the invention may be administered in single or multiple doses and for any length of time until the desired level of comfort is achieved. The person of ordinary skill in the art will recognise that the dosage amount, dosage regime and length of treatment will depend on factors such as, for example, the level of discomfort, the location of the discomfort and the health of the subject. In the case of a lens solution, the composition may be administered once a day (when the contact lens is applied). In the case of eye drops, the composition may be administered every half hour or hourly, up to, for example, eight times a day. In the case of oral administration, the composition may be administered, for example, in single or multiple (e.g. three) doses, once or more than once (e.g. three times) a day.

It has been found by the present inventors that a useful dose of omega-3 compound is between about 100 and 3000 mg per day. For example, the dose of omega-3 compound may be between about 100 and 750 mg per day (e.g. 750 mg per day), between about 100 and 300 mg per day, or about 150 mg per day. Where both an omega-3 and an omega-6 compound are administered, a useful dose is between about 100 and 200 mg per day of omega-3 compound and between about 50 and 100 mg per day of omega-6 compound. For example, the dose of omega-3 and omega-6 compound is 150 mg and 50 mg per day, respectively.

The composition may be administered in the form of one or more unit doses. For example, three capsules having a combined dose of 750 mg of omega-3 compound, or a precursor thereof, may be administered once a day to achieve a desired daily dose of omega-3 compound.

The ophthalmic device containing an omega-3 compound may be prepared by contacting a solution containing the omega-3 compound with the ophthalmic device. The omega-3 compound may be contacted with the ophthalmic device prior to selling or otherwise delivering the ophthalmic device to a subject (e.g. adding the omega-3 compound to a solution prior to sealing the package, and subsequently sterilizing the package) or during the preparation of the ophthalmic device. As outlined above, in one embodiment, the omega-3 compound is incorporated into liposomes which are attached to the device (such as a lens) and which then permit the omega-3 compound to be released during wearing of the device.

Sterilization can take place at different temperatures and periods of time. Sterilization is preferably carried out using filter sterilization.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds an omega-3 compound, or a precursor thereof, or a pharmaceutical composition which is effective for treating the condition and may have a sterile access port (for example the container may be an intravenous solution bag or a vial having a stopper pierceable by a hypodermic injection needle). The label or package insert indicates that the omega-3 compound, or precursors thereof, or the pharmaceutical composition is used for treating the condition of choice. In one embodiment, the label or package insert includes instructions for use and indicates that the therapeutic composition can be used to improve comfort or ameliorate decrease in ocular comfort during wearing of a contact lens/ophthalmic device.

The kit may comprise (a) an omega-3 compound, or a precursor thereof, or a pharmaceutical composition; and (b) a second container comprising a solution that is suitable for application to the eye, carriers, excipients, other active ingredients and the like. The kit in this embodiment of the invention may further comprise one or more package inserts. The inserts may, for example, indicate that the omega-3 compound, or precursors thereof, or the pharmaceutical composition and the component contained in the second container can be used to improve comfort or ameliorate decrease in ocular comfort during wearing of a contact lens/ophthalmic device, and provide instructions for use of the kit. The second container may comprise a solution that is suitable for application to the eye (e.g. an aqueous solution) and/or pharmaceutically-acceptable buffers, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may also comprise (a) an omega-3 compound, or a precursor thereof, or a pharmaceutical composition; and (b) a contact lens. The kit in this embodiment of the invention may further comprise one or more package inserts. The inserts may, for example, indicate that the omega-3 compound, or a precursor thereof, or the pharmaceutical composition and the contact lens can be used to improve comfort or ameliorate decrease in ocular comfort during wearing of a contact lens/ophthalmic device, and provide instructions for use of the kit.

One skilled in the art will recognize many methods and materials similar or equivalent to those described herein, which could be used in the practice of the present invention. The present invention is in no way limited to the methods and materials described.

The present invention will now be more fully described with reference to the accompanying example and drawings. It should be understood, however, that the description following is illustrative only and should not be taken in any way as a restriction on the generality of the invention described above.

### Examples

To assess the improved comfort achieved by the invention, 45 individuals were selected who did not suffer from dry eye disorder, conjunctivitis, glaucoma, recent eye surgery or other irritation or trauma or other allergy-related eye discomfort. For the purposes of the study, the participants were asked to rate the awareness of the lens, comfort and perceived lack of “dryness” after having worn contact lenses on three scales as follows:
(a) 1-100, where 50 is “no lens awareness”;
(b) 1-100, where 100 is “totally comfortable eyes”; and
(c) 1-100, where 100 is “no ocular dryness”.

Each parameter was rated by participants twice a day, the first time about two hours after waking and where lenses had not been worn during the night or prior to assessment. The second rating was taken in the evening, more specifically upon noticing symptoms beginning, or just before going to sleep if no symptoms were observed.

All participants were provided with Acuvue Advance™ (galyfilcon A) lenses. They were worn daily and disposed of afterwards. For the omega-3 compound, participants were supplied with commercially available capsules containing omega-3. Three capsules were taken each morning (preferably with food) for a maximum of 65 days (for 35 (±3) days wearing no vision correction, or normal vision correction if required; seven days wearing no vision correction or only glasses if any vision correction needed; up to 10 days wearing no vision correction or glasses if any vision correction needed; and up to 10 days wearing contact lenses). Each dose of three capsules contained three grams of fat but less than 10 mg cholesterol. In particular, this dosage contained EPA (450 mg), DHA (300 mg), and flaxseed oil (1000 mg).

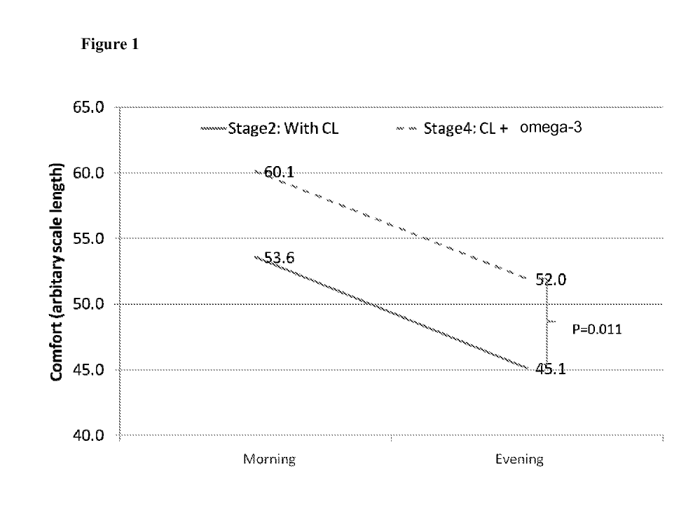

FIG. 1 shows that the group administered the omega-3 compounds recorded a consistently higher level of comfort both prior to applying a lens each morning and at the end of the day, wearing the lens. The difference of about seven is statistically significant, with a p value of 0.011.

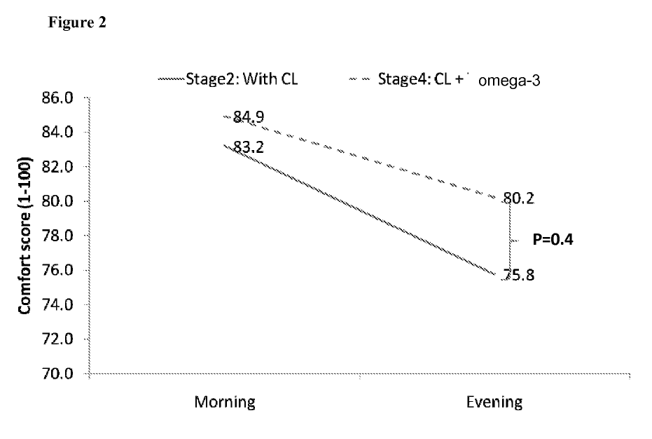

In FIG. 2, the “comfort score” is represented. Here it can be seen that the difference prior to lens wear was smaller but by evening there was still a higher comfort score from those administered the commercially available supplement (with a p value of 0.4).

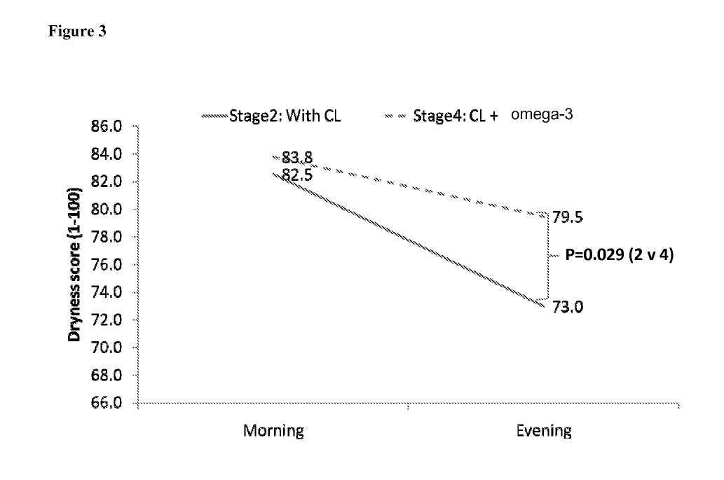

FIG. 3 records the scores for ocular dryness. Again, the scores in the morning prior to lens wear were relatively similar (although slightly higher in the omega-3 group). However, by evening, there was a significant difference in the average scores, with those administered the omega-3 compound being significantly higher (with a p value of 0.029).

It will be understood that the invention disclosed and defined in this specification extends to all alternative combinations of two or more of the individual features mentioned or evident from the text or drawings. All of these different combinations constitute various alternative aspects of the invention.

What is claimed is:

1. A method of preventing ocular discomfort, improving ocular comfort or ameliorating decrease in ocular comfort during wearing of an ophthalmic device, the method comprising administering to a person desiring to wear the ophthalmic device, a composition containing a therapeutically effective amount of an omega-3 compound, or a precursor thereof.

2. A method according to claim 1, wherein the omega-3 compound, or precursor thereof, includes one or more of alpha linolenic acid (ALA), eicosapentaenoic acid (EPA) and docosahexaenoic acid (DHA).

3. A method according to claim 1, wherein the person does not have, prior to this administration, any one or more of (1) dry eye disorder, (2) recent eye surgery or other irritation or trauma, (3) conjunctivitis, (4) glaucoma or (5) another allergy related eye discomfort.

4. A method according to claim 1, wherein the person is first assessed as being in need of improved ocular comfort or ameliorated decrease in ocular comfort during wearing of an ophthalmic device.

5. A method according to claim 1, wherein the person is not taking an omega-3 supplement to their normal diet prior to the administration.

6. A method according to claim 1, wherein the ophthalmic device is a contact lens.

7. A method according to claim 1, wherein the administration is topical directly to the eye.

8. A method according to claim 1, wherein the administration is oral.

9. A method according to claim 8, wherein the omega-3 compound, or a precursor thereof, is administered at a dose of between about 100 and 3000 mg per day.

10. A method according to claim 9, wherein the omega-3 compound, or a precursor thereof, is administered at a dose of between about 100 and 750 mg per day.

11. A method according to claim 10, wherein the omega-3 compound, or a precursor thereof, is administered at a dose of about 750 mg per day.

12. A method according to claim 8, wherein the composition is administered in the form of one or more unit doses.

13. A method according to claim 12, wherein the composition is administered in three unit doses.

14. A method according to claim 1, wherein the composition is administered for a period of about four to eight weeks prior to wear of the ophthalmic device.

15. A method according to claim 1 wherein an omega-6 compound, or a precursor thereof, is also administered.

16. A method according to claim 1, wherein no omega-6 compound, or precursor thereof, is administered.

17. A method according to claim 1, wherein the composition consists essentially of an omega-3 compound, or a precursor thereof.
